(19) United States
(12) Patent Application Publication
(10) Pub. No.: US 2025/0304708 A1
(43) Pub. Date: Oct. 2, 2025

(54) TREATMENT OF CANCER BY BLOCKADE OF OSTEOPROTEGERIN

(71) Applicant: THE REGENTS OF THE UNIVERSITY OF CALIFORNIA, OAKLAND, CA (US)

(72) Inventors: Anil Bhushan, San Francisco, CA (US); Yao Wang, Shenzhen (CN)

(21) Appl. No.: 19/109,853

(22) PCT Filed: Sep. 7, 2023
(86) PCT No.: PCT/US2023/032185

§ 371 Date: Mar. 7, 2025

Related U.S. Application Data
(60) Provisional application No. 63/374,854, filed on Sep. 7, 2022.

(51) Int. Cl. C07K 16/28; A61P 35/00

(52) U.S. Cl. CPC C07K 16/2878; A61P 35/00

(57) ABSTRACT

The disclosure provides, in various aspects, methods of treating cancer in a subject in need of treatment therefor, comprising administering to the subject a therapeutically effective amount of an osteoprotegerin inhibitor, wherein the osteoprotegerin inhibitor inhibits osteoprotegerin binding to TRAIL. In further aspects, the disclosure provides methods of achieving a therapeutic outcome in a subject in need of treatment, wherein the method comprises the administration to the subject of a treatment which inhibits OPG-mediated suppression of tumor immunity.

### CROSS-REFERENCE TO RELATED APPLICATIONS

This application claims the priority benefit under 35 U.S.C. § 119 (e) of U.S. Provisional Application No. 63/374,854, filed Sep. 7, 2022, which is incorporated herein by reference in its entirety.

### STATEMENT OF GOVERNMENT INTEREST

Not applicable.

### BACKGROUND

Stromal-rich cancers such as breast cancer, pancreatic cancer, colorectal and esophageal cancer, respond poorly to current immunotherapy approaches and generally have poor prognosis. It is now increasingly accepted that tumor progression does not only depend entirely on genetic and epigenetic defects of tumor cells alone, but is in large part controlled by the tumor microenvironment (TME). The TME or stroma consists of cells from endothelial, mesenchymal, and hematopoietic origins embedded in an extracellular matrix (ECM).

One element of the TME is what are referred to as cancer-associated fibroblasts (CAFs). CAFs are a heterogeneous collection of fibroblast cells. In healthy tissues, fibroblasts are well known for their role in building, maintaining and remodeling the ECM. Fibroblasts are also known for their role in immune surveillance, activating immune cells and modulating immune responses. In cancer, diverse populations of CAFs have been identified, and it is thought that they may be key regulators in the activation and suppression of tumor immunity. Accordingly, there is a need in the art for a better understanding of how CAFs might promote immunosuppression in cancer, and there is a need in the art for novel intervention to inhibit CAF-mediated suppression of tumor immunity, for the treatment of cancer. The foregoing needs are addressed by the novel methods of the invention disclosed herein.

### SUMMARY

By extensive experimentation, the inventors of the present disclosure have identified a key stromal immune regulator that can be targeted to block suppression of tumor immunity. Specifically, the inventors of the present disclosure have identified a subset of stromal fibroblasts that secrete osteoprotegerin (OPG), and have further determined that OPG directly blocks the cytotoxic activity of T-cells. Furthermore, the inventors of the present disclosure have demonstrated that interventions which block OPG prevents immunosuppression in the TME, resulting in T-cell activation to promote tumor removal.

Osteoprotegerin or OPG is also known as osteoclastogenesis inhibitory factor, or tumor necrosis factor receptor superfamily member 11B (TNFRSF11B). OPG is a cytokine receptor of the tumor necrosis factor (TNF) superfamily. The protein is produced by the TNFRSF11B gene. OPG is a secreted, soluble protein with cytokine-like activity. One known function of OPG is to act as a decoy receptor for the ligand (RANKL) of Receptor Activator of Nuclear Factor kappa-B (RANK). In this context, OPG inhibits RANK-RANKL interactions and suppresses osteoclasts and bone resorption. Another known function of OPG is to act as a decoy ligand for TNF-related apoptosis-inducing ligand (TRAIL). TRAIL can bind to Death Receptor 4 and Death Receptor 5 extracellular domains displayed by infected cells or tumor cells, and initiate apoptosis. Secreted OPG binds TRAIL and thus blocks TRAIL-mediated apoptosis.

In the context of cancer, TRAIL-based therapeutic approaches have been explored, based on either an administration of TRAIL-receptor (TRAIL-R) agonists or a recombinant TRAIL. These approaches, however, seem to elicit a limited therapeutic efficacy. OPG, which acts as a decoy and blocks TRAIL-receptor interactions has not been explored in the clinical setting. Bone-derived OPG has been shown to increase survival of breast cancer cells that reach the bone microenvironment as part of the metastatic process. OPG is also known as a pro-angiogenic agent driving the vascularization of tumors. However, to the knowledge of the inventors of the present disclosure, OPG-mediated immunosuppression has not been previously discovered or proposed. Herein is described the identification of a subset of CAFs, iCAFs, are present in the TME and secrete OPG. Secreted OPG can play a role in directly inhibiting FasL and TRAIL utilized by T effector cells for their cytotoxic activity. In this way secreted OPG by iCAFS construct an immunosuppressive state within the TME. The inventors of the present disclosure have advantageously discovered that certain CAFs, herein termed OPG-iCAFs, are present in the TME and tumor stroma and secrete OPG.

Meanwhile, cytotoxic T-Cells can remove infected cells and other aberrant cells such as cancer cells. Cytotoxic T-cell recognition of target cells is mediated by T-cell receptor (TCR) binding to target antigens presented by MHC on the surface of the target cell. Whether or not the T-cell then initiates a cytotoxic response against the target cell is determined by additional engagement of the cytotoxic inducing machinery present on the cell surface. The two main ligand-receptor pairs that mediate the cytotoxic function are FasL-Fas and TRAIL-DR4/5. Accordingly, the cytotoxic activity of T cells relies on both target recognition and engagement of the apoptosis signaling to initiate clearance.

As disclosed herein, the inventors of the present disclosure have determined that OPG in the tumor stroma acts to inhibit TRAIL-mediated apoptosis. This was discovered to be the result of action by a subset of CAFs present in the tumor stroma that secrete OPG into the TME. The OPG binds to TRAIL in sufficient amount to inhibit TRAIL-mediated cytotoxic activity of T-cells. Thus, when T-cells encounter target cells, the pro-apoptotic interaction of TRAIL with Death Receptors on the target cell is blocked by bound OPG. The inventors of the present disclosure have determined that this suppressive action is a key mechanism by which cancer cells escape immune clearance.

The inventors of the present disclosure have further determined that inhibition of OPG binding to TRAIL has dual therapeutic effects. First, inhibiting OPG's TRAIL binding activity rescues the ability of T-cells to effectively target and destroy cancer cells by initiating TRAIL-mediated apoptosis. Second, the inventors of the present disclosure have unexpectedly discovered that inhibition of OPG results in a reduction in OPG-releasing CAFs, and thus promotes durable tumor immunity. The foregoing discoveries provide the art with various novel methods as disclosed herein.

In a first aspect, the scope of the invention encompasses a novel method treating cancer in a subject by the administration of an OPG-inhibiting agent to the subject. In one aspect, the scope of the invention encompasses methods of treating highly stromal cancers such as breast, pancreatic, and esophageal cancer. In another aspect, the scope of the invention encompasses a method of blocking OPG-mediated suppression of tumor immunity. In another aspect, the scope of the invention encompasses a method of removing immunosuppressive CAFs from the TME. The various inventions are described in detail next.

### BRIEF DESCRIPTION OF THE DRAWINGS

FIG. 1 shows a schematic of immune mediated target cytotoxicity.

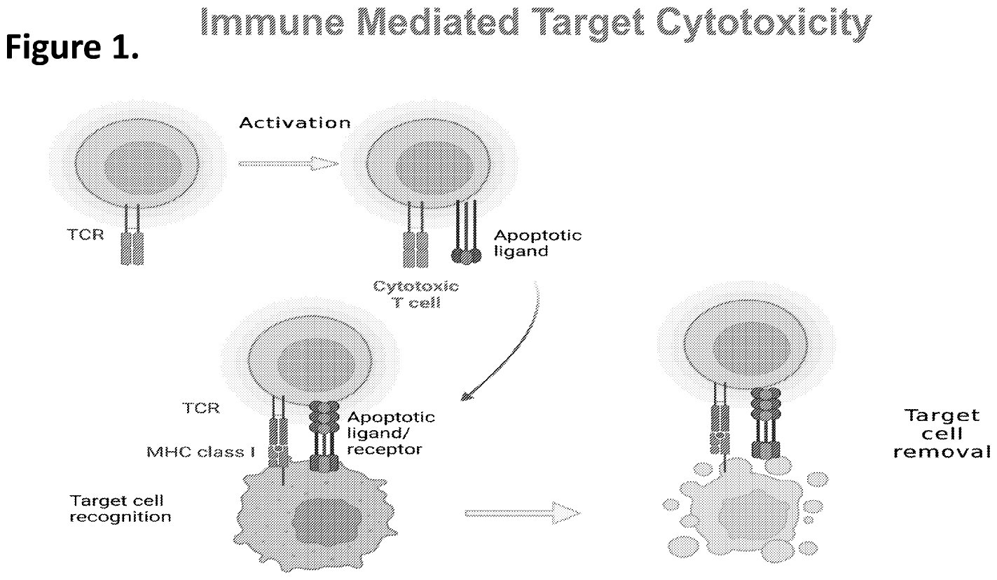

FIG. 2 shows that CAFs subset suppresses T cells.

FIG. 3 shows results of cell sorting to isolate various CAFs and other cell types following tumor dissection from breast cancer model (EO771 implanted in the fat pad). apCAFs are represented in cluster 2, iCAFs are represented in cluster 3, and myCAFs in cluster 4.

FIG. 4 shows that iCAFs suppress T cell activity.

FIG. 5 shows that an anti-OPG antibody restores T cell function.

FIG. 6 shows OPG in the stromal compartment in cancer.

FIG. 7 shows that an anti-OPG antibody shrank a tumor.

FIG. 8 shows that blocking OPG inhibited tumor growth in pancreatic cancer models.

### DETAILED DESCRIPTION

The inventions disclosed herein provide the art with various methods of inhibiting CAF-mediate suppression of tumor immunity. In various implementations, the scope of the invention encompasses the following general methods:
a method of inhibiting CAF-mediated suppression of tumor immunity in a subject;
a method of reducing the abundance of immunosuppressive CAFs in the TME of a subject;
a method of promoting immune clearance of tumor cells in a subject;
a method of promoting T-cell mediated cytotoxic removal of cancer cells in a subject;
a method of promoting TRAIL-mediated cytotoxic removal of cancer cells in a subject; and
a method of treating cancer in a subject; wherein
the method comprises the administration to the subject of a treatment which inhibits OPG-mediated suppression of tumor immunity. In a primary embodiment, the treatment comprises the administration of an OPG-inhibiting agent. The various elements of the foregoing general methods are described next.

Subjects. The methods of the invention are applied to a subject. As used herein, a “subject” may be an animal of any species. The subject may be a human or a non-human animal such as a test animal, livestock, pet, or veterinary subject. Exemplary animals include human beings, non-human primates, cats, dogs, mice, rats, cows, pigs, horses and others. The scope of the invention further extends to the treatment of cancer cells in vitro, for example, explanted or cultured cells treated with an OPG-inhibiting agent in any context, including for the identification, screening, or evaluation of OPG-inhibiting agents. The subject will typically be a subject in need of treatment for cancer. A subject in need of treatment for cancer may be, for example, a subject at risk of developing cancer, a subject with precancerous tissues, a subject having been diagnosed with cancer, a subject that has had cancer previously, for example being at risk of remission, and a subject suspected of having cancer by one or more diagnostic indicia.

Cancer. The methods of the invention are applied in various aspects of cancer treatment, including administration to a subject in need of treatment for cancer. As used herein “cancer” may encompass any neoplastic condition, including, for example, breast cancer, bladder cancer, brain cancer, cervical cancer, colorectal cancer, esophageal cancer, gastric cancer, head and neck cancer, kidney cancer, lung cancer, leukemia, lymphoma, myeloma, ovarian cancer, pancreatic cancer, prostate cancer, sarcoma, and skin cancer, including melanoma. The cancer may be primary cancer, metastatic cancer, or any other type.

In a primary implementation, the cancer is a stroma-rich cancer, i.e. a solid tumor or a tumor having a high stroma content. Stroma is typically present in all cancers, with some, such as lymphoma, having minimal stroma, and some, such as solid tumors, having very high stromal content, e.g. over 90%. Stroma may comprise various elements, including, for example, extracellular matrix, fibroblasts, CAFs, mesenchymal stromal cells, osteoblasts, and other cellular and acellular elements of tumor stroma, as known in the art. Tumor Stroma Ratio is a diagnostic measure of stroma content, as known in the art, measured as the proportion of tumor tissue relative to surrounding stromal tissue, as observed in sectioned tissue. In various embodiments, the high-stroma cancer is a tumor having greater than 50% stroma. In other embodiments, the high-stroma cancer is cancer of a type known to typically have a high stroma content, for example, of breast, pancreatic and esophageal.

Tumor Microenvironment. As used herein, tumor microenvironment of TME refers to the cells surrounding or associated with a neoplastic growth, as known in the art. The TME comprises a diverse set of non-malignant cells and non-cellular elements of the tumor niche, tumor surroundings, or otherwise tumor-associated cells, tissues, and structures.

Treatment. The inventions disclosed herein encompass various methods of treating cancer. As used herein, “treatment” encompasses any therapeutic intervention against cancer, including, for example, inhibiting tumor growth, facilitating the death and removal of cancer cells, reducing tumor size, slowing or halting the progression of cancer, inhibiting metastasis, reducing the risk of remission, ameliorating the symptoms of cancer, preventing cancer onset or recurrence, increasing immune action against cancer, inhibiting suppression of cancer immunity, inhibiting or reducing the abundance of OPG-secreting CAFs, or any other therapeutically beneficial outcome.

Administration. The treatments of the invention encompass the administration of agents to subject, for example, the administration of an OPG-inhibiting agent. The methods of the invention encompass the administration of agents in a therapeutically effective amount. As used herein, a therapeutically effective amount means an amount sufficient to induce a measurable biological effect (including cumulative effects from multiple administrations. Exemplary biological effects include increased T-cell reactivity to cancer cells, increased TRAIL signaling, reduced OPG binding to T-cell receptors, killing of cancer cells, blockade of OPG-mediated T-cell suppression, reduction of OPG-secreting CAFs, reducing tumor size, inhibiting tumor progression, and other therapeutic effects.

The therapeutically effective dose that will be administered may vary significantly depending on the nature of the agent, for example, based on the type of agent (small molecule or biologic), half-life in the body, effective dose required for effect at the target tumor(s), and route of administration. Exemplary doses in the range of 10 nanograms per dose to 200 grams per dose may be used, for example 10 ng, 100 ng, 500 ng, 1 mg, 10 mg, 50 mg, 100 mg, 200 mg, 500 mg, or 1 g. For example, monoclonal antibody therapeutics are typically administered at doses of 1-100 mg per kg body weight, with some antibodies administered at even higher doses, such as obinutuzumab which may be administered at 1-2 grams per kg body weight. The doses may be administered according to a suitable schedule, for example, daily, weekly, monthly, or at various intervals between doses as suitable for the selected agent and cancer status of the subject.

Administration route will vary according to the agent and may encompass, for example, any of systemic administration, local administration, peritumoral administration, and intratumoral administration. Exemplary administration routes include oral, intravenous, intraperitoneal, subcutaneous, subdermal, intradermal, and others as suitable for the selected agent and target tumor.

OPG Inhibitors. The scope of the invention encompasses the administration of an OPG inhibitor. As used herein, an “OPG inhibitor” is any composition of matter that, directly or indirectly, inhibits OPG binding to TRAIL. The inhibition of this binding may be achieved by any mechanism, for example, including blocking TRAIL-binding motifs of OPG, disrupting TRAIL-OPG binding, competitive inhibition, non-competitive inhibition, and uncompetitive inhibition.

Antibodies. In a primary implementation, the OPG inhibitor is an antibody or fragment thereof. The antibody or fragment thereof will comprise one or more amino acid sequences imparting binding affinity for TRAIL, e.g. complementarity-determining regions (CDRs) or like compositions. The antibody may disrupt OPG-TRAIL binding by any mechanism. In one implementation, the antibody binds to OPG such that the TRAIL binding motif, is partially or wholly sterically blocked from interacting with TRAIL. The TRAIL binding domain of OPG is believed to comprise the c-terminal D5 and D6 OPG domains comprising amino acids 195-319, encompassing sequences with homology to death domain 4 (amino acids 198-269) and homology Death Domain 5 (amino acids 270-365).

The antibody may comprise any OPG binding antibody known in the art or fragments thereof which have OPG binding ability, e.g. CDRs or other OPG-binding elements of an anti-OPG antibody. Various anti-OPG antibodies are known in the art.

Exemplary anti-OPG antibodies include anti-OPG antibodies and binding compositions disclosed in United States Patent Application Publication Number 20190352413, and in “Anti-OPG Antibodies,” by De Arbeau Carvalho et al.; U.S. Pat. No. 6,919,433, “Monoclonal antibodies that Bind OCIF,” by Goto et al.

Experimental uses of anti-OPG antibodies are described in: Arnold et al., 2019, A therapeutic antibody targeting osteoprotegerin attenuates severe experimental pulmonary arterial hypertension, Nature Communications 10, Article number: 5183; Riches et al., Skladal et al., 2005. Investigation of osteoprotegerin interactions with ligands and antibodies using piezoelectric biosensors, 2009, BioSensors and Bioelectronics 20:2027-2034; and Waterman et al., 2007, The antibody MAB8051 directed against osteoprotegerin detects carbonic anhydrase II: Implications for association studies with human cancers, Int. J. Cancer: 121, 1958-1966.

Multiple commercially available anti-OPG antibodies are available, including, for example: NB100-56505 and NBP1-51670 (Novus Biochem); AM06539SU-N(Origene); GTX82749 (Genetex); LS-C169286 (Lifespan Biosciences); PA5-34946, PA5-86053, MA5-15715, MA5-15960, MA5-34922, MA5-34923, and MA5-15726 (Invitrogen Antibodies); AM06539SU-N, SM7070P, AM06550SU-N, DM2005 and DM2018 (Acris Antibodies, GmbH); BF0156 (Affinity Biosciences); MAB10335, MAB6241, MAB12971, H00004982-K, and MAB3414 (Abnova Biosciences); OAAD00388 (Aviva Systems Biology); EM1701-98 and EM1701-99 (HUABIO Research); ALX-804-813-C100, ALX-804-813B-C100, ADI-AAM-020-E, and ALX-804-532-C100 (Enzo Life Sciences), 139256 and 104289 (NovoPro Bioscience); 10-6001 (Abeomics); AO1471a, AO1482a, and ALS12119 (Abgent); 3448-1 and ab124820 (RabMAbs); abx011830 and abx016009 (Abbexa); 030691, 030692, and O8065-04 (United States Biological); MOB-1442z-S(P), CBMAB-O0022-CQ, CBMAB-O0026-CQ, CBMAB-O0472-CQ, CBMAB-O0473-CQ, CBMAB-O0474-CQ, CBMAB-O0475-CQ, CBMAB-O0881-CQ, CBMAB-O0884-CQ, CBMAB-O0888-CQ, CBMAB-T3072-YJ, MOB-1442z, MOB-1507CT, MOR-3609, MRO-1136—CN, NEUT-2118CQ, and ZG-0330C (Creative BioLabs); 10271-R340 (Sino Biologica); 98A1071 and 40938 (Active Motif); 119-12729 (Ray Biotech); and APR08890G (Leading Antibody).

Many of the foregoing antibodies are mouse, rabbit, rat or other non-human antibodies raised against human OPG protein. The anti-OPG antibodies and fragments thereof of the invention encompass chimeric or humanized forms of the foregoing antibodies and other modified forms thereof suitable for use in humans. For example, chimeric antibodies may be produced by fusing the antigen binding region of a non-human antibody with specificity against OPG with a human or otherwise immunogenically acceptable constant domain. Similarly, humanized antibodies may be generated by replacing the hypervariable loops of a fully human antibody with the hypervariable loops of a non-human antibody with specificity against OPG. The antibodies may be produced recombinantly. Chimeric or humanized antibodies may be produced by various methods known in the art, including CDR grafting to human or humanized scaffolds, for example, the trastuzumab scaffold.

In one embodiment, the OPG-inhibiting antibody comprises a whole, or substantially whole, antibody. As known in the art, a whole antibody comprises a dimer, each dimer comprising a heavy chain and a light chain. The heavy chain comprises three constant regions, C_{H2} and C_{H3}, below the hinge, and C_{H1} above the hinge. The heavy chain also comprises a variable region V_{H} which comprises framework sequences that present the three heavy chain CDRs. The light chain comprises one constant region C_{L}, which is paired with C_{H1}, and a variable region V_{L}, comprising framework sequences which contain and present the three light chain CDRs.

In alternative implementations, the OPG inhibitor comprises an antibody fragment, for example, a Fab (Fragment, antigen binding), for example, an antibody fragment which retains OPG binding and inhibiting ability. In such implementations, the fragment will comprise a subset one or more parts of a whole antibody, depending on the particular fragment configuration of the composition. In one embodiment, the antibody fragment is a Fab (product of papain cleavage) or a Fab′ (product of pepsin cleavage), comprising C_{H}1, V_{H}, C_{L}, and C_{L} sequences and lacking the FC portion of the antibody. In one embodiment, the composition is a F(ab′)2 fragment, comprising a Fab′ dimer. In one embodiment, the OPG inhibitor comprises a single chain variable fragment (scFv), comprising a fusion protein of the VH and VL sequences of an antibody, wherein the chain sequences are joined by a linker sequence, for example, a linker of 10-50 amino acids, for example about 20-25 amino acids, for example, comprising all or a majority of glycine, serine, and/or threonine. For example, in one embodiment the OPG inhibiting agent is a single-chain variable fragments (scFv) comprising the heavy and light chains of the OPG-binding variable region of an antibody linked by a short amino acid spacer or disulfide bond. In one embodiment the OPG inhibiting agent is a diabody comprising two scFv fragments associated in a bivalent dimer, including a homodimer or bispecific heterodimer. Triabodies and tetrabodies may be used as well.

The OPG inhibiting antibody may comprise a hybrid composition derived from multiple antibody sequences, or may comprise novel engineered sequences. Any scaffold format is within the scope of the invention as well, comprising any amino acid sequence that can orient and present the CDRs to achieve OPG binding, include non-immunoglobulin protein scaffolds, synthetic antibody mimetics, and other compositions, as known in the art. The scaffold body compositions of the invention may comprise any type of immunoglobulin. The antibodies or immunoglobulins of the invention may comprise any Ig isotype, for example, IgG, IgA, IgD, IgE and IgM forms. The IgG sequences may comprise any isoform, for example, IgG1, IgG2, IgG3 and IgG4 from human or other animal sources.

Additional OPG-Inhibiting Compositions. In another implementation, the OPG inhibitor of the invention comprises an alternative composition that selectively binds to OPG and prevents its binding to TRAIL or otherwise disrupts OPG inhibition of TRAIL activity. The OPG-binding composition may be any composition, for example, a designed ankyrin repeat protein (DARPin), an aptamer, an affibody, a knottin, an affilin, an affimer, an anticalin, an atrimer, an avimer, a centyrin, a fynomer; a Kunitz domain, an obody, a pronectin, or a repebody.

Small Molecules. The scope of the invention further encompasses small molecules that disrupt OPG-TRAIL interactions.

Pharmaceutical Compositions. It will be understood that the OPG inhibitors maybe be administered in any format or formulation, as known in the art. For example, the OPG inhibitor may be formulated with, functionalized with, incorporated in, or otherwise combined with any number of additional components to improve efficacious delivery thereof, including for example, carriers, excipients, drug delivery vehicles, targeting moieties, preservatives, additional active agents (combination products), and other drug delivery compositions, devices, or packaging known in the art.

### Diagnostic Methods

As disclosed in the Examples below, the inventors of the present disclosure have advantageously determined that OPG is elevated in subjects having stromagenic tumors amenable to the treatment methods of the invention. Accordingly, the scope of the invention encompasses methods of assessing a cancer subject for OPG abundance to determine if the subject is amendable to treatment by administration of an OPG inhibitor.

The general diagnostic method comprises the steps of:
obtaining a sample from a subject, wherein the subject is a cancer subject;
measuring the abundance of OPG in the sample;
comparing the measured value of OPG abundance to a normal or cutoff value, or by other statistically relevant tools, determining if the subject has an elevated abundance of OPG,
wherein, if the subject has an elevated abundance of OPG, the subject is deemed amendable to treatment with an OPG inhibitor.

The subject may be a subject having cancer or suspected of having cancer. The sample may be any sample indicative of OPG abundance in stromal compartment of cancer in the subject. Surprisingly, the inventors of the present disclosure have determined that OPG is elevated in the blood of animals having stromagenic cancer, providing a convenient means of monitoring relevant OPG abundance. In one embodiment, the sample is a blood sample, or blood serum isolated therefrom. In other embodiments, the sample is another bodily substance, for example, urine, saliva, sweat or other sample type. In one embodiment, the sample is cellular material obtained from a biopsy.

OPG abundance may be measured by any method known in the art. Direct measurement of OPG in the sample may be achieved by known techniques, for example immunofluorescent staining, chromatography, mass spec, colorimetric assays, and other detection techniques for quantifying analytes in a sample. Indirect measurement of OPG may be achieved by measurement of OPG proxies, such as iCAF abundance or products of OPG metabolism.

The determination of elevated OPG may be achieved by comparing the measured value to representative values for OPG abundance in normal subjects or subjects not having a stromagenic cancer and representative subjects having stromagenic cancers. Any statistically relevant tool for classification or discrimination analysis of measured values may be used, for example, establishing thresholds for elevated OPG, for example, by cutoff values indicative of elevated OPG. For example, a blood concentration of OPG greater than a selected threshold may be indicative of elevated OPG, for example, the threshold being any of 500, 1,000, 2,000, 3,000, 4,000, 5,000, 6,000, 7,000, 8,000, 9,000, 10,000, 12,000, or 15,000 μg/ml blood. Alternatively, a classifier model may be generated, which may be applied to measured OPG abundance data to classify the subject's normal or elevated OPG status. In another embodiment, a predictive model is generated wherein the input is the measured abundance of OPG and the output is the likelihood of the subject being amenable to OPG-inhibition treatment. Thresholds and classification schemes may be implemented with selected specificity and sensitivity parameters, as known in the art. The thresholds, classifier models, or predictive models may be generated by any suitable statistical tools known in the art. For example, the relationship between measured OPG value and amenability to anti-OPG treatment may be established using statistical methods such as: logistic regression analysis, linear discriminate analysis, partial least squares-discriminate analysis, multiple linear regression analysis, multivariate non-linear regression, backwards stepwise regression, threshold-based methods, tree-based methods, generalized additive models, supervised and unsupervised learning models, cluster analysis, and other statistical model generating methods known in the art.

In one implementation, the method comprises the further step of administering an OPG inhibitor to the subject if the subject is found to have elevated OPG.

In a related implementation, OPG abundance is used to monitor or assess the efficacy of an anti-cancer treatment administered to a subject, for example, as a companion diagnostic, including administration of OPG-inhibitors, or any other cancer treatment known in the art, for example, immunotherapy, chemotherapy, radiotherapy, or surgery. The general method encompasses the process of: obtaining at least a first and a second sample from a subject receiving a cancer treatment, the first and second samples being obtained at two different time points; measuring the abundance of OPG in the sample; determining if OPG abundance is decreasing over time, wherein, if OPG abundance is decreasing over time, the cancer treatment is deemed to be effective.

The scope of the invention further encompasses kits for the detection and quantification of OPG in a sample. The kit will comprise at least one detection agent for quantification of OPG (or a proxy thereof) in the sample, for example, a labeled antibody and the kit will contain one or more other components, for example, commonly packaged with the OPG detection agent, for example, OPG standards, secondary antibodies, containers, sample collection tubes, instructions for use, software for quantification and statistical analysis, and other elements useful for achieving measurement of OPG in a sample.

### EXAMPLES

### Example 1

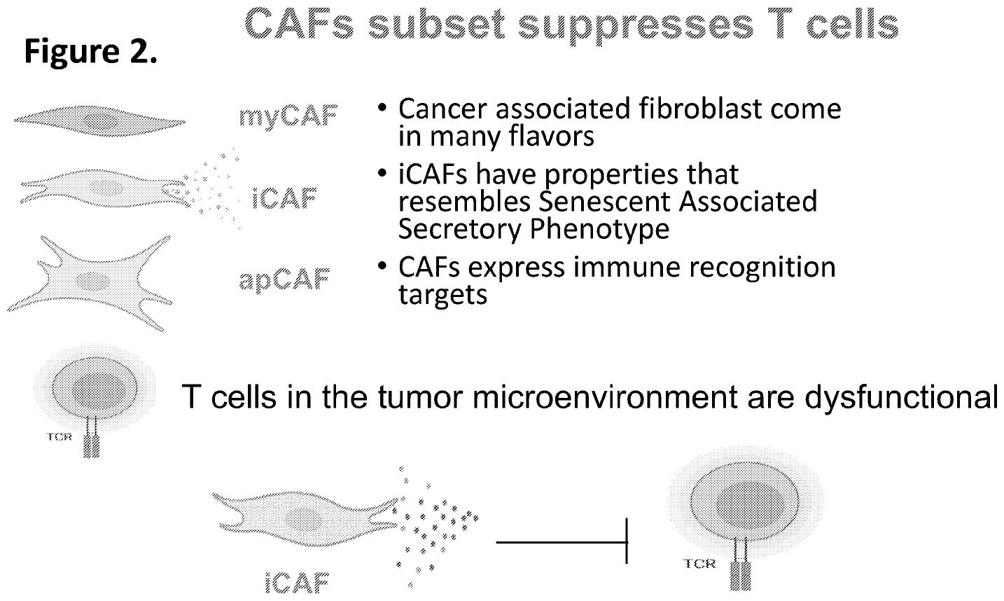

Identification of CAFs as Mediators of Immune Evasion. Various populations of CAFs are known to be found in the tumor stroma, as summarized in FIG. 2. Myofibroblasts (myCAFs) express alpha-smooth muscle actin (α-SMA), indicative of activated fibroblasts. Inflammatory CAFs (iCAFs) are an inflammatory CAF phenotype, secreting cytokines such as IL-6 and CXCL12, and often identified by expression of the marker Ly6C. Antigen-presenting CAFs (apCAFs) are characterized by expression of MHC-II and PGE₂ and promotion of regulatory T cells (Tregs).

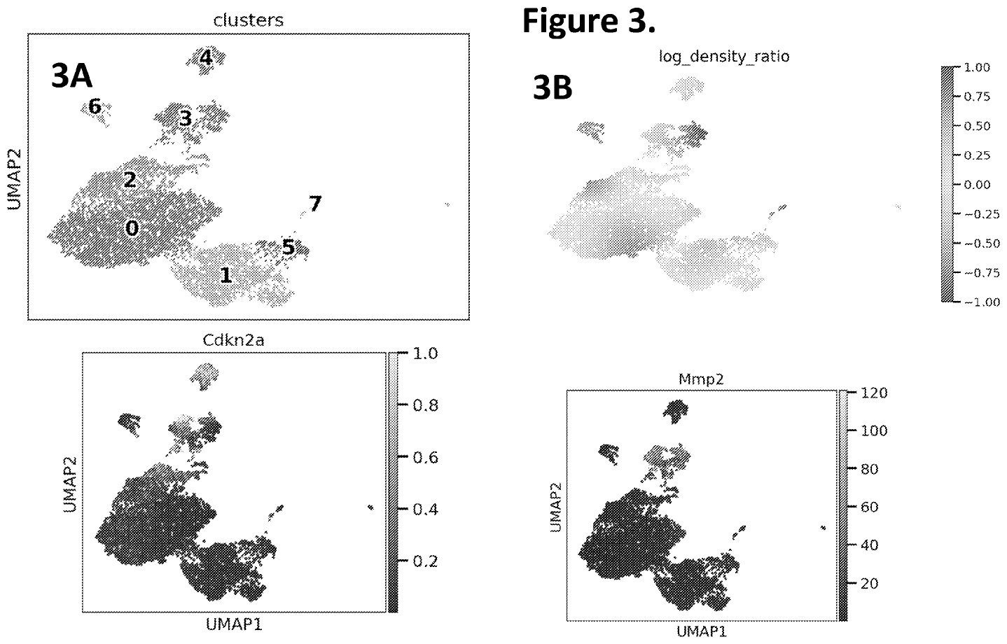

Tumor dissection from breast cancer model (EO771 implanted in the fat pad) and cell sorting was used to isolate various CAFs and other cell types, as depicted in FIG. 3. apCAFs are represented in cluster 2, iCAFs are represented in cluster 3, and myCAFs in cluster 4. Single cell transcriptome analysis revealed that the iCAFs express high levels of OPG, indicating that this factor may play a role in stroma-induced tumor immunity.

### Example 2

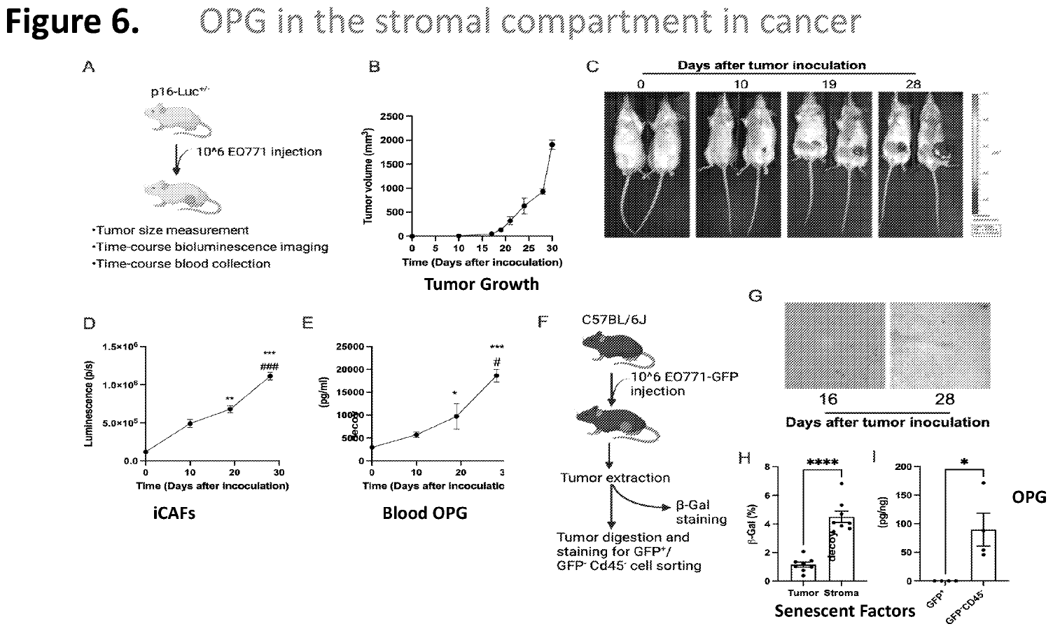

OPG Abundance increases with Growth of High Stroma Tumor. Luciferase knockin mouse p16^{LUC+/-} mouse model enables visualization and quantification of tumors stroma by luciferase signal generated in senescent stromal cells surrounding tumors. p16^{LUC+/-} mice were each injected with 10⁶ EO771 cells, a mouse syngeneic model of mammary adenocarcinoma, introduced into the hindquarters. Tumor volume and OPG blood levels were monitored (FIG. 6A). Tumor volume increased significantly over 28 days, as depicted in FIGS. 6B and 6C (left: representative control mouse; right: EO771 injected mouse). Luminescence surrounding the tumor, indicative of tumor stromal senescence increased in parallel (FIG. 6D). OPG blood concentration also increased in concert with tumor growth (FIG. 6E). These results demonstrate a substantial increase in OPG is associated with growth of the tumor and surrounding stroma.

### Example 3

ICAFs and OPG in Tumor Stroma. In another set of experiments, C57BL/J mice were injected with GFP-expressing EO771 cells (FIG. 6F). At 16 days and 28 days following tumor inoculation, tumors were extracted, digested, and sorted on GFP expression, luciferase expression, and CD45 expression to isolate tumor cells (GFP+) from stromal cells (Luc+, GFP−, CD45−). Tumor and stromal cells were cultured. Cultures were analyzed for β-Gal, a marker of senescent phenotype. FIG. 6H shows substantial β-Gal expression by stromal cells, indicative of increased iCAF abundance. FIG. 6I depicts OPG secretion by cultured cells, wherein stromal cells produced high amounts of OPG, while tumors produced none. These results demonstrate that OPG-secreting iCAFs are present and active in tumor stroma.

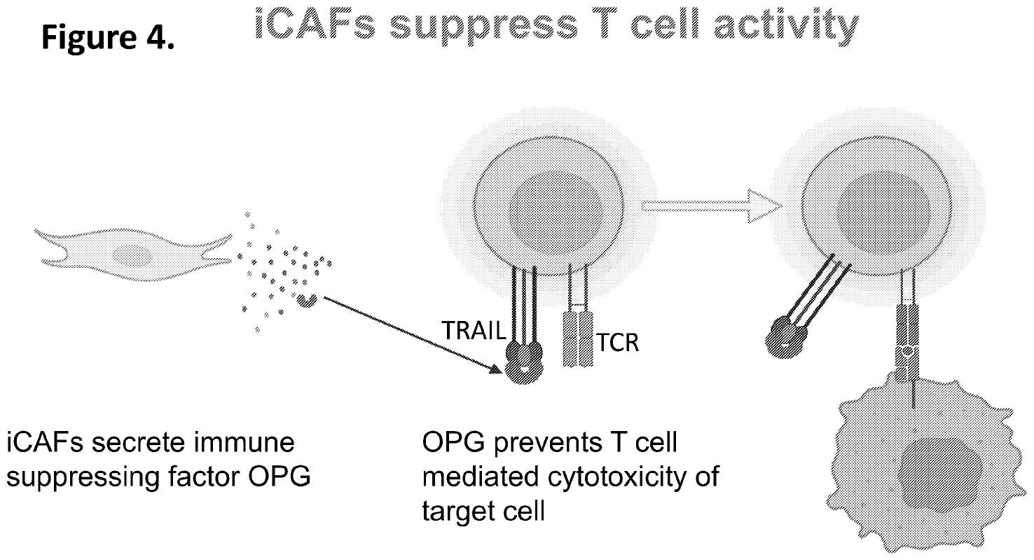

Based on the foregoing results it was hypothesized that OPG, a known ligand of TRAIL, was inhibiting immune surveillance by blocking TRAIL binding of target ligands on tumor cells, as summarized in FIG. 4.

### Example 4

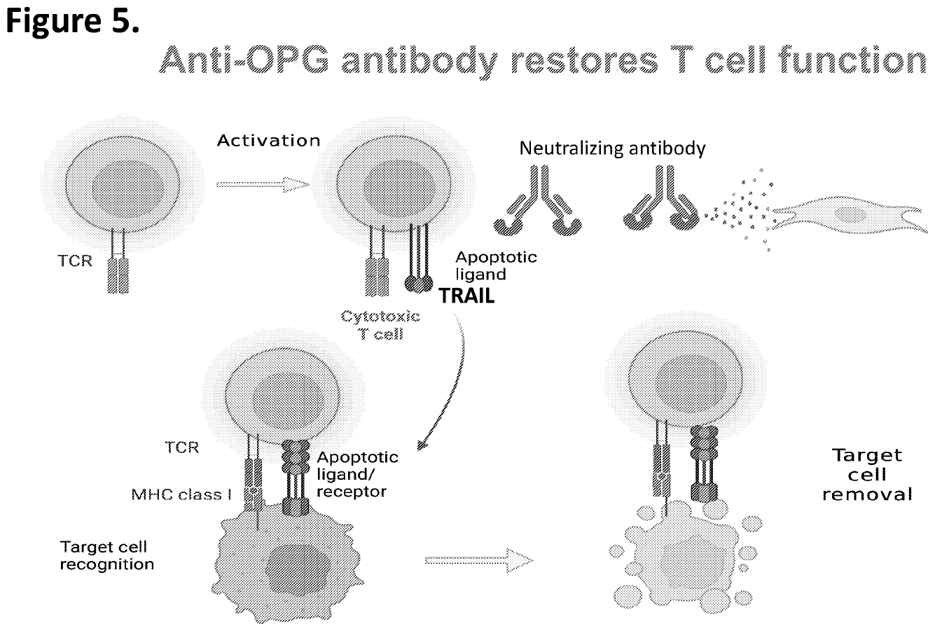

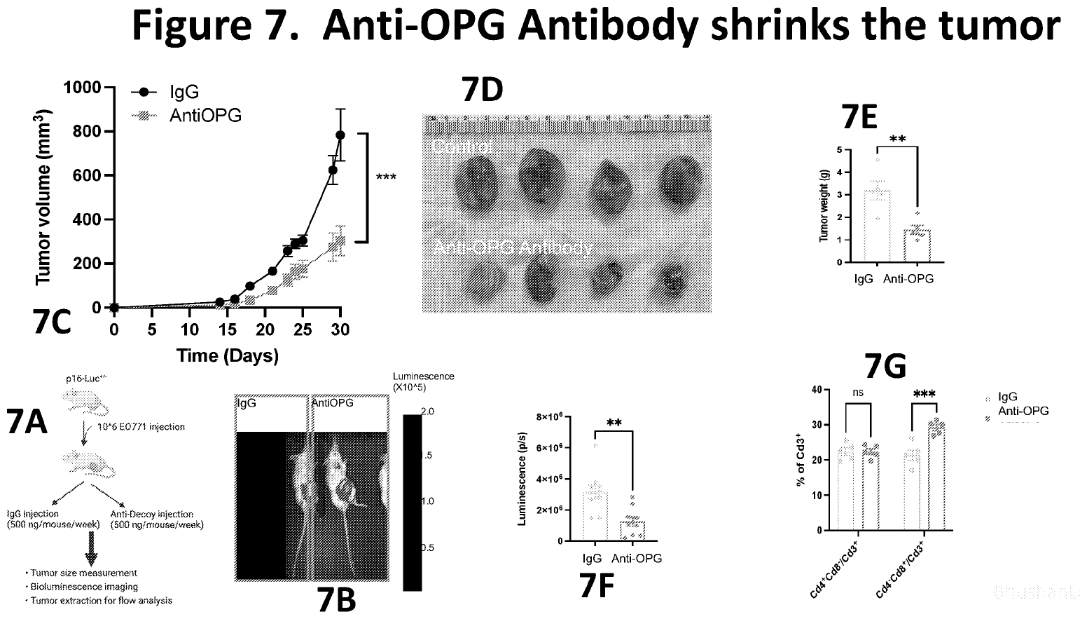

Treatment with Anti-OPG Antibody Reduces Tumor by Cytotoxic T-Cell Activity in Breast Cancer Model. p16^{LUC+/-} mice were injected with EO771 cells as above. One set of the p16^{LUC+/-} mice were treated with an anti-OPG antibody, Mouse Anti-Osteoprotogerin/TNSRSF11 antibody catalog number AF459 by R&D Systems (Minneapolis, MN), and a control set was injected with an isotype IgG control. Injections were administered commencing at the time of tumor inoculation and were administered weekly thereafter (FIG. 7A), with control Ig or anti-OPG antibody being administered at 500 ng per mouse per injection. Tumor size was monitored for four weeks by caliper measurements while mice were scruffed and conscious. As depicted in FIG. 7B, tumor size, was reduced in anti-OPG antibody treated mice relative to control mice. As depicted in FIG. 7C, tumor volume was significantly reduced over time in the treated mice relative to control mice. At four weeks, tumors were removed, weighed (7C), and dissociated for cellular analysis. FIG. 7D shows that tumors were significantly and substantially reduced in size in mice treated with the anti-OPG antibody, as measured by tumor volume at four weeks. Likewise, tumor weight at four weeks was substantially reduced by anti-OPG antibody treatment (FIG. 7E). FIG. 7F shows that stromal cells (Luc+, GFP−, CD45−) were substantially reduced in anti-OPG antibody-treated mice. FIG. 7G shows that activated cytotoxic T-cells were significantly increased in anti-OPG antibody-treated mice [CD8+/CD4−/CD3+]. Notably, similar results were obtained using other anti-OPG antibodies including those generated using human peptide sequences (Data not shown). These results demonstrate that treatment with an OPG inhibitor comprising an anti-OPG antibody will effectively reduce tumor size and promote tumor removal by cytotoxic T-cells, for example, as depicted in FIG. 5.

### Example 5

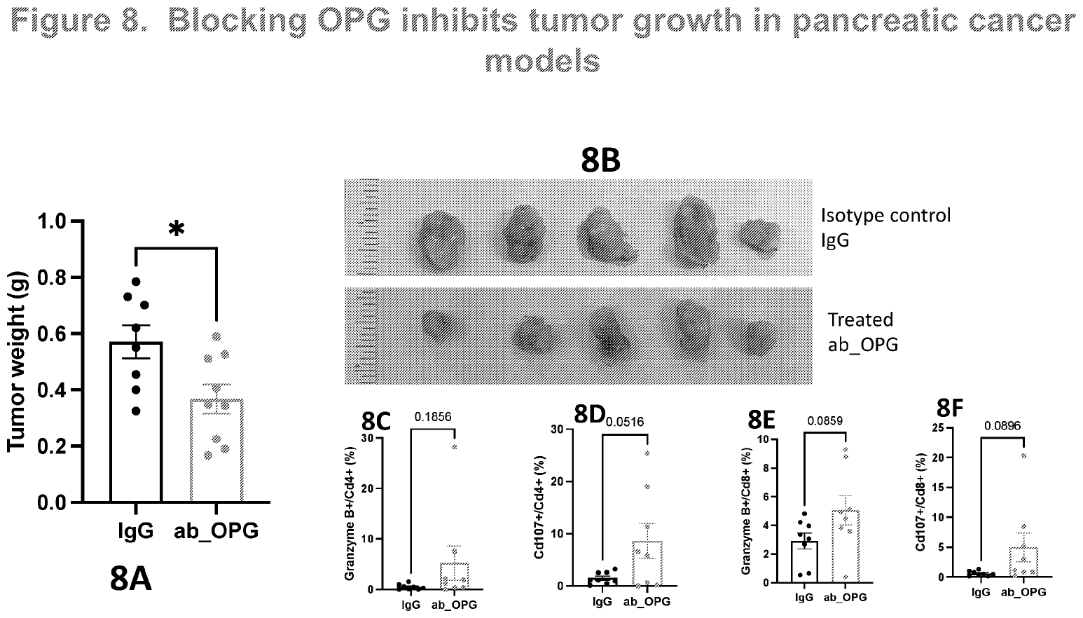

Treatment with Anti-OPG Antibody Reduces Tumor by Cytotoxic T-Cell Activity in Breast Cancer Model. The experiments of Example 4 were repeated using a mouse model of pancreatic cancer. Results are summarized in FIG. 8. FIG. 8A shows that tumor weight at 4 weeks was significantly reduced in mice treated with anti-OPG antibody. FIG. 8B shows significantly reduced tumor size in treated mice compared to control mice at four weeks. Additionally, CD4+ and CD8+ cells were assayed for expression of Granzyme B and CD107, classical markers of cytotoxic T-Cell infiltration. Both CD107 and granzyme B expression was significantly increased in anti-OPG antibody-treated mice, in both CD4+ and CD8+ cells (FIGS. 8C-8F), demonstrating that treatment with anti-OPG antibody reduced tumor size by relieving cytotoxic T-cell suppression.

Similar results to those described above were obtained with other anti-OPG antibodies, for example, human anti-osteoprotogerin/TNFRSF11B antibody catalog number AF805 from R&D Systems (Minneapolis, MN).

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

What is claimed is:

1. A method of treating cancer in a subject in need of treatment therefor, comprising
administering to the subject a therapeutically effective amount of an osteoprotegerin inhibitor, wherein the osteoprotegerin inhibitor inhibits osteoprotegerin binding to TRAIL.

2. The method of claim 1, wherein
the cancer is selected from the group consisting of breast cancer, bladder cancer, brain cancer, cervical cancer, colorectal cancer, esophageal cancer, gastric cancer, head and neck cancer, kidney cancer, lung cancer, leukemia, lymphoma, myeloma, ovarian cancer, pancreatic cancer, prostate cancer, sarcoma, and skin cancer.

3. The method of claim 1, wherein the cancer is a stromagenic cancer.

4. The method of claim 1, wherein the osteoprotegerin inhibitor comprises an antibody or fragment thereof which binds to osteoprotegerin and inhibits its interaction with TRAIL.

5. A method of achieving a therapeutic outcome in a subject in need of treatment,
wherein the therapeutic outcome is selected from the group consisting of:
reducing the abundance of immunosuppressive CAFs in the TME of a subject;
promoting immune clearance of tumor cells in a subject;
promoting T-cell mediated cytotoxic removal of cancer cells in a subject;
promoting TRAIL-mediated cytotoxic removal of cancer cells in a subject; and
a method of treating cancer in a subject; wherein
the method comprises the administration to the subject of a treatment which inhibits OPG-mediated suppression of tumor immunity.

6. The method of claim 5, wherein treatment comprises the administration of an OPG-inhibiting agent.

7. The method of claim 6, wherein the OPG-inhibiting agent comprises an antibody or fragment thereof which binds to OPG and inhibits its capacity to interact with TRAIL.
